(19) United States
(12) Patent Application Publication
(10) Pub. No.: US 2026/0248087 A1
(43) Pub. Date: Aug. 27, 2026

(54) GENERATING DOUBLED HAPLOID PLANTS

(71) Applicant: Syngenta Crop Protection AG, Basel (CH)

(72) Inventors: Gavin John Hall, Bracknell Berkshire (GB); Ian Jepson, RTP, NC (US); Weiguo Liu, Research Triangle Park, NC (US); Michal Jakub Zawadzki, Bracknell Berkshire (GB); Melissa Catherine Brazier-HIcks, Bracknell Berkshire (GB)

(73) Assignee: Syngenta Crop Protection AG, Basel (CH)

(21) Appl. No.: 19/148,129

(22) PCT Filed: Jan. 23, 2024
(86) PCT No.: PCT/US2024/012500

§ 371 Date: Jul. 15, 2025

Related U.S. Application Data
(60) Provisional application No. 63/482,081, filed on Jan. 30, 2023.

(51) Int. Cl. A01H 1/08; A01H 1/02; A01H 5/10; A01H 6/46

(52) U.S. Cl. CPC A01H 1/08; A01H 1/026; A01H 5/10; A01H 6/4684

(57) ABSTRACT

Provided herein are methods for generating doubled haploid plants. The methods include obtaining a haploid plant tissue, contacting the haploid plant tissue with a safener compound, contacting the haploid plant tissue with a chromosome doubling agent, and regenerating a doubled haploid plant. The safener compound (e.g., metcamifen) and the chromosome doubling agent (e.g., colchicine) are applied sequentially or simultaneously. Additionally, the methods include further contacting the haploid plant tissue with a vigor response compound (e.g., thiamethoxam) in which the vigor response compound and chromosome doubling agent are applied sequentially or simultaneously.

### FIELD OF THE INVENTION

The present invention relates to plant breeding and efficient methods to produce doubled haploid plants.

### BACKGROUND

The production of doubled haploid (DH) plants is important and beneficial to breeders. Once a haploid inducer plant is used to produce a haploid plant or embryo, said haploid plant or embryo must then be converted into a doubled haploid. Typical methodologies for producing DH plants require extensive resources. There is a need for improved methods to reduce the burden and improve the efficiency of DH production. One way to obtain DH plants is by treating a haploid plant with a doubling agent (e.g., colchicine). Colchicine and other chemicals convert haploid plants into DH plants during cell division by inhibiting spindle assembly and blocking cell wall formation. However, the use of colchicine and many doubling agents is stressful, toxic, and can be lethal to embryos and seedlings.

Thus, promoting haploid stress tolerance can improve the production efficiency of the DH process. Improving stress tolerance will allow greater survival throughout the process.

Here, we showcase new strategies demonstrating that selected chemistries can be used to induce a protective mechanism of the plants for better protection of the haploids from stresses during DH production processes, leading to increased haploid survival and haploid-to-doubled-haploid conversion rate, and therefore DH production efficiency.

### SUMMARY

To reduce the burden and improve the efficiency of generating a doubled haploid plant, disclosed herein is a method comprising obtaining a haploid plant tissue, contacting the haploid plant tissue with a safener compound, contacting the haploid plant tissue with a chromosome doubling agent, and regenerating a doubled haploid (DH) plant. In this method, the safener compound and the chromosome doubling agent are applied sequentially or simultaneously. The haploid plant tissue is further contacted with a vigor response compound, which can be applied with the chromosome doubling agent sequentially or simultaneously. Additionally, in this method, the safener compound and the vigor response compound can be applied sequentially or simultaneously. The vigor response compound is selected from the group consisting of thiamethoxam and saponins. The safener compound is selected from the group consisting of metcamifen, benoxacor, dichlormid, isoxadifen, cloquintocet-mexyl, fenclorim, and cyprosulfamide. The chromosome doubling agent is selected from the group consisting of colchicine, trifluralin, pronamide, dithipyr, nitrous oxide, and oryzalin. The haploid plant tissue in this method can be maize, wheat, rice, barley, sunflower, soybean, watermelon, cucumber, tomato, pepper or brassica. When the haploid plant tissue is from maize, the haploid plant tissue can be an ear, a microspore, callus tissue, or an embryo.

Another method of this invention includes generating a doubled haploid plant comprising obtaining a haploid plant tissue, contacting the haploid plant tissue with a vigor response compound, contacting the haploid plant tissue with a chromosome doubling agent, and regenerating a doubled haploid plant. Contact with the vigor response compound can occur sequentially or simultaneously to contact with the chromosome doubling agent.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the discloses compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “allele” refers to a variant or an alternative sequence form at a genetic locus. In diploids, a single allele is inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

As used herein, the term “biomarker” refers to a measurable indicator of a different biological state or condition, including stress tolerance. These biomarkers can include, for example, GST27, glycosyltransferases, and cytochrome P450s.

As used herein, the term “chromosome doubling agent” means a chemical that doubles the number of chromosomes in a cell (eg. From haploid to diploid or diploid to tetraploid, etc.) by blocking normal cell cycle division. Such agents are typically anti-microtubule agents such as colchicine, pronamide, dithipyr, trifluralin, or another known anti-microtubule agent). Those skilled in the art are familiar with compounds that can cause chromosome doubling.

As used herein, the term “doubled haploid embryo” refers to an embryo that has one or more cells that contain 2 sets of homozygous chromosomes. The related term “doubled haploid” means a plant cell, tissue or plant derived from a haploid. The diploid has two sets of chromosomes (2n) and is typically homozygous. The “doubled haploid is developed by doubling the haploid set of chromosomes by means of using a “chromosome doubling agent”. As used herein, the term “embryo” refers to the embryo formed after one sperm nucleus from a pollen grain fuses with the egg cell to create a diploid (2N) embryo. Diploid (2N) embryos may become haploid (1N) embryos by genome elimination. Haploid (1N) embryos may also form after failed sperm fertilization or sperm-egg fusion where there is some stimulation of the egg to develop directly into a haploid embryo via parthenogenesis.

As used herein, the term “endosperm” refers to the tissue formed after one sperm nucleus from a pollen grain fuses with the polar nuclei sac to create a triploid (3N) endosperm.

As used herein, the term “escape rate” refers to the percent of false haploids due to non-marker-expression of diploids from all colorless embryos (true haploids plus false haploids). As used herein, an “escape” refers to a diploid that goes undetected through the haploid selection process.

As used herein, the term “gene” refers to a hereditary unit including a sequence of DNA that occupies a specific location on a chromosome and that contains the genetic instruction for a particular characteristic or trait in an organism.

As used herein, the term “genotype” refers to the genetic constitution of a cell or organism. As is known in the art, a genotype can relate to a single locus or to multiple loci, whether the loci are related or unrelated and/or are linked or unlinked. In some embodiments, a genotype is expressed in terms of a haplotype (defined herein below).

As used herein, the term “germination” refers to the sprouting of an embryo usually after a period of dormancy. Germination is dependent upon appropriate environmental conditions, such as temperature, water, and oxygen. The embryo may be intact as part of a seed or isolated and cultured on artificial media in sterile conditions.

As used herein, the term “germplasm” refers to the totality of the genotypes of a population or another group of individuals (e.g., a species). The term “germplasm” can also refer to plant material; e.g., a group of plants that act as a repository for various alleles. The phrase “adapted germplasm” refers to plant materials of proven genetic superiority; e.g., for a given environment or geo-graphical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

As used herein, the term “haploid” means a plant cell, tissue or plant having a set of (n) chromosomes. In a haploid organism, only half of the normal number of chromosomes are present.

As used herein, haploid induction rate (“HIR”) means the number of surviving haploid kernels or embryos over the total number of kernels or embryos after an ear is pollinated with haploid inducer pollen or after a haploid inducer ear is pollinated with wild type pollen.

As used herein, the term “heterotic group” refers to a group of genotypes or inbred lines that demonstrate similar heterotic response when crossed with genotypes or inbred lines from other genetically distinct germplasm groups. There is a closer degree of genetic relationship of lines contained within a heterotic group versus the more distant degree of genetic relationship of lines compared between heterotic groups. In general, the hybrid of two inbred lines crossed together within the same heterotic group shows much less heterosis than the hybrid of an inbred line from one heterotic group crossed to an inbred line from a different heterotic group. A particular heterotic group can include multiple lines having diverse genetics.

As used herein, the term “hybrid” refers to a plant that is the offspring of genetically dissimilar parents produced by crossing plants of different lines or breeds or species, including but not limited to the cross between two inbred lines (e.g., a genetically heterozygous or mostly heterozygous individual).

As used herein, the term “locus” refers to a position (e.g., of a gene, a genetic marker, or the like) on a chromosome of a given species.

As used herein, “maternal haploid inducer” refers to a line that produces pollen and, when crossed as a male, results in the gynogenic development of haploid seeds. A “paternal haploid inducer” refers to a line that when used as a female in a cross, results in androgenic development of haploid seeds. A haploid inducer plant can use either of these maternal or paternal mechanisms to derive haploids and can be generically referred to as an “inducer” or “haploid inducer” without specifying the mechanism of a particular line.

As used herein, the term “plant” can refer to a whole plant, any part thereof, or a cell or tissue culture derived from a plant. Thus, the term “plant” can refer to any of: whole plants, plant components or organs (e.g., leaves, stems, roots, etc.), plant tissues, seeds and/or plant cells, unless otherwise specified.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). For example, in the case of a haploid plant evaluated for SCD, phenotype can refer to fertile pollen shed and/or a seed obtained via pollination with that pollen. In other cases, a phenotype is the result of interactions among several genes, which in some embodiments also results from an interaction of the plant and/or plant cell with its environment.

As used herein, the term “potted rate” or “potting rate” refers to the percent of germinated embryos successfully transferred to soil from total treated haploid embryos. The “potted rate” is used as an indication of plant health in monitoring the success of recovery and growth after treatment of haploids. As used herein, the term “transplant rate” refers to the survival of healthy plants after embryos were germinated and transferred to soil.

As used herein, the terms “progeny” and “progeny plant” refer to a plant generated from a vegetative or sexual reproduction from one or more parent plants. In maternal haploid induction, the desired seeds on the female parent are haploids, thus not progeny of the inducing haploid line. The progeny of the haploid seed is not the only progeny. There may also be hybrid (diploid) seed and a subsequent plant grown therefrom, and further progeny of the hybrid backcrossed with the female plant. Additionally, progeny may include aneuploid progeny (comprising an uneven mix of chromosomes from the parent plants), which are typically discarded. Both the haploid seed and the hybrid seed can be progeny. A progeny plant can be obtained by cloning or selfing a single parent plant, or by crossing two or more parental plants. For instance, a progeny plant can be obtained by cloning or selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1s, F2s, and the like. An F1 can thus be (and in some embodiments is) a hybrid resulting from a cross between two breeding parents, while an F2 can be (and in some embodiments is) a progeny resulting from self-pollination of the F1 hybrids.

As used herein, the term “regenerate,” and grammatical variants thereof, refers to the production of a plant from tissue culture.

As used herein, the term “safener” refers to a chemical compound used in combination with herbicides to reduce the effect of herbicide damage on crop plants.

As used herein, the term “trait” refers to a phenotype of interest, a gene that contributes to a phenotype of interest, as well as a nucleic acid sequence associated with a gene that contributes to a phenotype of interest.

### DETAILED DESCRIPTION

The following description and examples recite various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

One aspect of the invention provided herein is a method of generating a doubled haploid plant comprising obtaining a haploid plant tissue, contacting the haploid plant tissue with a safener compound, contacting the haploid plant tissue with a chromosome doubling agent, and regenerating a doubled haploid plant. In one embodiment, the safener compound and the chromosome doubling agent are applied sequentially or simultaneously. In another embodiment, the haploid plant tissue is further contacted with a vigor response compound. In another embodiment, the vigor response compound and chromosome doubling agent are applied sequentially or simultaneously.

In a further embodiment, the safener compound is contacted with the haploid plant tissue prior contacting the haploid plant tissue with a chromosome doubling agent. In another embodiment, the safener compound is contacted with the haploid plant tissue for 24 hours to 168 hours prior to contact with the chromosome doubling agent. In another embodiment, the safener compound is at a concentration of 5-50 mg/L. In another embodiment, the safener compound contact occurs at 4-8° C. In yet another embodiment, the safener compound contact occurs at 6° C.

In one embodiment, the safener compound is contacted with the haploid plant tissue concurrently with contacting the haploid plant tissue with a chromosome doubling agent. In another embodiment, the safener compound is contacted with the haploid plant tissue concurrently with contact with the chromosome doubling agent for 24 hours to 48 hours. In yet another embodiment, the safener compound is at a concentration of 400-800 mg/L. In another embodiment, the safener compound and chromosome doubling agent contact occurs at 24-32° C. In still another embodiment, the safener compound and chromosome doubling agent contact occurs at 28° C.

In one embodiment, the safener compound is contacted with the haploid plant tissue after contacting the haploid plant tissue with a chromosome doubling agent. In an embodiment, the safener compound is contacted with the haploid plant tissue after contact with the chromosome doubling agent for 8-504 hours and the safener compound is at a concentration of 0-20 mg/L. The safener compound contact may occur at 24-32° C., and in another embodiment, the contact occurs at 28° C.

In an embodiment, the safener compound and the vigor response compound of the method are applied sequentially or simultaneously. The haploid plant tissue of the method may be from maize, wheat, rice, barley, sunflower, soybean, watermelon, cucumber, tomato, pepper, or brassica. In an embodiment, the haploid plant tissue is maize and may be an ear, a microspore, callus tissue, or an embryo. In an embodiment, the maize plant tissue is an embryo.

The safener compound of the method of is selected from the group consisting of metcamifen, benoxacor, dichlormid, isoxadifen, cloquintocet-mexyl, fenclorim, and cyprosulfamide. In an embodiment, the safener compound is metcamifen. In an embodiment, the chromosome doubling agent is selected from the group consisting of colchicine, trifluralin, pronamide, dithipyr, nitrous oxide, and oryzalin. In yet another embodiment, the chromosome doubling agent is colchicine. In one embodiment, the vigor response compound is selected from the group consisting of thiamethoxam and saponins. In another embodiment, the vigor response compound is thiamethoxam.

In one embodiment, the haploid plant tissue is contacted with thiamethoxam prior to colchicine treatment. In another embodiment, the thiamethoxam is applied at 10-1000 mg/L. In yet another embodiment, the thiamethoxam is applied for 24 to 48 hours. In another embodiment, the haploid plant tissue is contacted with the thiamethoxam during the colchicine treatment. In still yet another embodiment, the thiamethoxam is applied at 100 to 8000 mg/L. In another embodiment, the thiamethoxam is applied for 24 to 48 hours. In a further embodiment, the haploid plant tissue is contacted with the thiamethoxam after the colchicine treatment. In yet another embodiment, the thiamethoxam is applied at 0.5-12 g/L. In another embodiment, the thiamethoxam is applied for 24-504 hours. In a further embodiment, all previously mentioned contact is performed in solid or liquid media.

Another aspect of the invention is a method of generating a doubled haploid plant comprising obtaining a haploid plant tissue, contacting the haploid plant tissue with a vigor response compound, contacting the haploid plant tissue with a chromosome doubling agent, and regenerating a doubled haploid plant. In one embodiment, the vigor response compound is selected from the group consisting of thiamethoxam and saponins. In another embodiment, the vigor response compound is thiamethoxam.

In another embodiment, contact with thiamethoxam occurs prior to contact with the chromosome doubling agent. In a further embodiment, the thiamethoxam is applied at 10-1000 mg/L. In yet another embodiment, the thiamethoxam is applied for 0.5-24 hours. In another embodiment, contact with thiamethoxam occurs simultaneously with contact with the chromosome doubling agent. In a further embodiment, the thiamethoxam is applied at 100-8000 mg/L. In yet another embodiment, the thiamethoxam is applied for 24-48 hours. In another embodiment, contact with thiamethoxam occurs after contact with the chromosome doubling agent. In yet another embodiment, the thiamethoxam is applied at 0.5-12 g/L. In still yet another embodiment, the thiamethoxam is applied for 24-504 hours.

### EXAMPLES

### Example 1: Metabolism of Colchicine in Corn Embryos

An experiment was completed to evaluate the uptake of metcamifen into corn embryos, whether colchicine is metabolized in the embryos, and if metcamifen increased the rate of colchicine metabolism. Corn embryos were co-treated with 500 ppm colchicine in the presence of 200 ppm, 1000 ppm or no metcamifen (control) for 24 or 48 hours and frozen. One biological replicate was represented by five embryos, and three biological replicates were used per co-treatment.

Prior to extraction, embryos were washed for 20 seconds in 80:20 (v/v) acetonitrile:H₂O to remove any colchicine and metcamifen on the surface. Each replicate was extracted in 0.5 ml methanol using a fast prep maceration method. Then, samples were diluted 1 in 10 prior to analysis by Liquid Chromatography Mass Spectrometry (LCMS). Samples were analyzed alongside matrix matched standards of colchicine and metcamifen. Metabolite investigations were performed to identify any metabolites.

Metcamifen could be detected in co-treated samples at both 24 and 48 hours. The amount of colchicine recovered after 48 hours was double the amount recovered at 24 hours. Further, the addition of metcamifen did not reduce the amount of colchicine recovered. Duration and metcamifen co-treatment increased the recovery amount of one colchicine metabolite (−14) that had undergone O-demethylation, however this metabolism did not lead to a decrease in colchicine recovery demonstrating that it was a minor component. Therefore, the use of metcamifen did not significantly interact or degrade the availability of colchicine for doubling the haploid chromosomes.

For the examples below, GST27 was used as a quick indicator for treatment effectiveness.

### Example 2: Metcamifen Pre-Treatment of Maize Ears

The effect of temperature was tested independently by comparing 6° C. and 28° C. for both 3 and 5 days with 150 PAR light. The 28° C. temperature resulted in bleached and dead embryos and as result, was dropped. The 6° C. temperature resulted in a normal, unbleached phenotype and was chosen for all subsequent testing of metcamifen pre-treatment of maize ears.

Maize ears were pre-treated with metcamifen prior to embryo isolation and before moving the embryos to colchicine-containing doubling media. In all experiments, doubling media is semi-solid, thickened with Gelzan, unless liquid media is specified. Two methods of delivering the safener at concentrations of 100 and 1000 mg/L were tested. The first method wrapped the ear in a paper towel that was wet with formulated metcamifen. The second method used a sprayer to deliver the formulated metcamifen onto the ear surface at 6° C. with 150 PAR light. GST27 was measured after metcamifen pre-treatment and again at the end of colchicine treatment. A low level of GST27 was induced (<10 ng/mg Total Soluble Protein (TSP)) at the end of the ear pre-treatment. Following ear pre-treatment, embryos were isolated and treated with colchicine for 48 hours. At the end of the colchicine treatment, GST27 induction results were increased but insignificant (see Table 1).

TABLE 1
GST27 induction results from pre-treatment of ears with
metcamifen at 100 and 1000 mg/L for 3 and 5 days at 6° C.

| | | GST27 (ng/mg of | | GST27 (ng/mg of | |
|---|---|---|---|---|---|
| | | TSP) before doubling | | TSP) after doubling | |
| Duration | Metcamifen | | Std | | Std |
| (days) | (mg/L) | Mean | Error | Mean | Error |
| 3 | 0 | 4 | 1 | 161 | 41 |
| | 100 | 4 | 1 | 156 | 23 |
| | 1000 | 3 | 1 | 178 | 47 |
| 5 | 0 | 2 | 1 | 192 | 23 |
| | 100 | 2 | 1 | 245 | 19 |
| | 1000 | 4 | 1 | 303 | 79 |

The addition of 0.5% Tween20 in the formulation with metcamifen at both 100 and 1000 mg/L was tested in combination with spraying corn ears as a pre-treatment prior to embryo isolation. The addition did not improve the resulting GST27 expression level over 4 days.

### Example 3: Metcamifen Pre-Treatment of Isolated Embryos

Embryos from maize ears were isolated and treated with metcamifen prior to a colchicine doubling treatment. Isolated embryos plated on filter paper were pre-treated with a formulation of metcamifen at concentrations of 0, 10, 100, and 1000 mg/L in Murashige and Skoog Medium (MS media) for a duration of 1, 3, 5, or 7 days at 6° C. with 150 PAR light. GST27 induction was measured after pre-treatment at each time point and again at the end of the colchicine treatment. Resulting embryos were germinated, and the percentage of embryos successfully transferred to soil (percent potted rate) was measured as an indicator of plant health and pre-treatment effectiveness. GST27 was induced by metcamifen at a medium level (<100 ng/ml of TSP), but higher than ear pre-treatment that was less than 10 ng/ml of TSP. Pre-treatment with metcamifen (10 mg/L) at 6° C. for 1 day resulted in the highest percent potted rate (see Table 2).

TABLE 2
GST27 induction and potted rate results from pre-treatment of embryos
with metcamifen in MS medium for different durations at 6° C.

| | Metcamifen | GST27 | | | |
|---|---|---|---|---|---|
| Pre-treatment | (mg/L) in | (ng/mg TSP) | | Potted Rate | |
| Days | MS media | Mean | Std Error | Mean | Std Error |
| 1 | 0 | 6 | 1 | 14% | 4% |
| | 10 | 9 | 0 | 46% | 15% |
| | 100 | 21 | 1 | 12% | 2% |
| | 1000 | 11 | 1 | 10% | 8% |
| 3 | 0 | 6.3 | 4 | 17% | 10% |
| | 10 | 14 | 8 | 38% | 24% |
| | 100 | 30 | 2 | 12% | 3% |
| | 1000 | 71 | 15 | 16% | 6% |
| 5 | 0 | 14 | 12 | 4% | 2% |
| | 10 | 37 | 12 | 4% | 4% |
| | 100 | 61 | 26 | 16% | 4% |
| | 1000 | 60 | 16 | 17% | 8% |
| 7 | 0 | 51 | 5 | 16% | 9% |
| | 10 | 75 | 5 | 27% | 3% |
| | 100 | 92 | 9 | 24% | 12% |
| | 1000 | 108 | 16 | 17% | 13% |

The addition of 10% DMSO in the metcamifen formulation at 10, 100, and 1000 mg/L in the MS media was tested to potentially increase the uptake of metcamifen. This test was done at temperatures of both 6° C. and 20° C. over a course of 0 to 4 days. There was no increase in GST27 expression with the 10% DMSO addition at either temperature. The metcamifen application at 10 mg/L at all time points continued to provide the best GST27 induction in comparison to the control and other doses, regardless of temperature.

Embryo pre-treatment with metcamifen at 10 mg/L was tested at temperatures of 6° C. and 20° C. for 1 day to measure the impact of temperature on embryo health. The 6° C. pre-treatment had a significantly higher potting rate than the control. The 20° C. pre-treatment caused low embryo survival during colchicine treatment that followed resulting in a 2% potted rate (see Table 3). Overall, the addition of metcamifen in the media for pre-treatment of isolated embryos resulted in higher GST27 induction than the co-treatment of ears done in Example 1 (see Tables 1 to 3).

TABLE 3
GST27 induction and potted rate results from pre-treatment
of embryos with 10 mg/L metcamifen in MS medium for
1 day at 6° C. and 20° C.

| Pre- | Metcamifen | GST27 (ng/mg TSP) | | | |
|---|---|---|---|---|---|
| treatment | (mg/L) in | before doubling | | Potted Rate | |
| temperature | MS media | Mean | Std Error | Mean | Std Error |
| 6° C. | 0 | 3.83 | 1.35 | 27% | 11% |
| | 10 | 8.83 | 2.14 | 62% | 7% |
| 20° C. | 0 | 42.3 | 18.1 | 2% | 1% |
| | 10 | 140 | 56 | 23% | 11% |

To optimize the metcamifen concentration, further experiments are conducted for pre-treating embryos with metcamifen for 24 hours. Concentrations ranging from 5 mg/L to 20 mg/L are tested. Following pre-treatment, the pre-treated embryos are subsequently treated with colchicine for 24 and 48 hours.

### Example 4: Metcamifen Co-Treatment of Isolated Embryos During Colchicine Doubling

Isolated embryos were treated with metcamifen during colchicine doubling (also referred to as co-treatment). Metcamifen was added in the colchicine co-treatment media (a.k.a. doubling media) as a formulation at concentrations of 0, 200, 400, 800, 1200 mg/L. Metcamifen concentrations tested for a 24-hour duration ranged from 0-1000 mg/L. Metcamifen concentrations tested for a 48-hour condition ranged from 0-1200 mg/L. Three to four genotypes representing two heterotic groups were tested.

Initial testing on three genotypes included co-treatment of metcamifen and colchicine in Gelzan media at 28° C. with 150 PAR light at concentrations of 0, 200, and 1000 mg/L to identify a range of working concentrations. The 200 and 1000 mg/L concentrations induced similar GST27 expression levels for both the 24- and 48-hour durations. The resulting induction was higher (400 ng/mg TSP GST27) than recorded with pre-treatments of ears or isolated embryos. Results from the 48-hour co-treatment demonstrated that 200 mg/L metcamifen increased the percent potted rate and provided better plant health and germination rate than the 1000 mg/L and control. Both concentrations of metcamifen (200 and 1000 mg/L) in the 24-hour co-treatment increased the percent potted rate along with an increase in the doubled haploid ears produced versus the control. Metcamifen co-treatment of isolated embryos optimization continued by testing levels of 0, 400, 600, and 800 mg/L for 24 and 48 hours with 4 genotypes across 2 heterotic groups. GST induction (ng/mg TSP) was measured after metcamifen co-treatment. While GST27 induction was observed with all co-treatments and higher than in the control, the highest induction was measured with 800 mg/L in the 48-hour co-treatment. One genotype yielded significantly higher GST27 induction at 800 mg/L for 48-hours than the rest. The percent potted rate after metcamifen co-treatment was similar for both time durations when sorted by genotype or heterotic group.

Both time durations tested offered benefits over the control with the best percent potting rate resulting from the 24-hour co-treatment (800 mg/L) while the highest mean GST27 induction rate resulted from the 48-hour co-treatment (1200 mg/L). Levels of metcamifen at 200, 400, and 800 mg/L for 24-hours and metcamifen at 800 and 1200 mg/L for 48-hours demonstrated evidence of GST27 induction and better potting rate compared to the colchicine only control (see Table 4).

TABLE 4
GST27 induction and potted rate results from co-treatment of embryos
with colchicine and 0-1200 mg/L metcamifen for 24-48 hours.

| | | GST27 Induction | | | |
|---|---|---|---|---|---|
| Duration | Metcamifen | (ng/mg TSP) | | Potted Rate | |
| (Hours) | (mg/L) | Mean | Std Error | Mean | Std Error |
| 24 | 0 | 139.75 | 27.97 | 78% | 4% |
| | 200 | 383.25 | 47.13 | 84% | 4% |
| | 400 | 396.13 | 68.84 | 84% | 4% |
| | 800 | 489.38 | 82.26 | 92% | 2% |
| 48 | 0 | 164.63 | 28.98 | 7% | 2% |
| | 400 | 609.50 | 63.34 | 14% | 7% |
| | 800 | 666.50 | 80.04 | 73% | 7% |
| | 1200 | 796.63 | 98.52 | 71% | 8% |

### Example 5: Metcamifen Post-Treatment During Germination

Doubled haploid embryos were treated with metcamifen during germination. Metcamifen was tested in the germination media at 0, 5, and 10 mg/L after colchicine treatment completion for 24 or 48 hours. The germination media comprised Gelzan at a concentration of 3.5 g/L. Four genotypes were tested representing two heterotic groups. GST27 expression at both 24 and 48 hours was higher than the control group. The percent potting rate was not increased as result of these post-treatments. It was observed that metcamifen induced fast root growth and the root tip was unable to penetrate the media. This led to a looping of the roots. A reduction in Gelzan concentration from 3.5 g/L to 2.5 g/L was tested (comparison data not presented here). The experiments were then carried out with 2.5 g/L Gelzan for metcamifen post-treatments while the control (0 metcamifen) contained 3.5 g/L Gelzan. Metcamifen was tested as 0, 5, 10 and 20 mg/L after colchicine treatment completion for 24 and 48 hours. The reduction in Gelzan of the metcamifen post-treatments led to a higher percent potting rate (see Table 5).

TABLE 5
Post-treatment of embryos with 0-20 mg/L metcamifen for
24-48 hours: effects on GST27 induction and potted rate

| Duration | Metcamifen | GST27 (ng/mg TSP) | | Potting Rate | |
|---|---|---|---|---|---|
| (Hours) | (mg/L) | Mean | Std Error | Mean | Std Error |
| 24 | 0 | 160 | 40 | 49% | 5% |
| | 5 | 327 | 30 | 69% | 5% |
| | 10 | 345 | 42 | 62% | 6% |
| | 20 | 404 | 34 | 51% | 7% |
| 48 | 0 | 204 | 35 | 13% | 4% |
| | 5 | 317 | 34 | 18% | 8% |
| | 10 | 290 | 34 | 24% | 6% |
| | 20 | 400 | 62 | 23% | 8% |

### Example 6: Thiamethoxam Co-Treatment During Colchicine Doubling

In addition to testing of safeners such as metcamifen, testing the effects of other compounds, such as thiamethoxam (TMX), were completed. Isolated embryos were co-treated with TMX during colchicine doubling in the doubling media. The co-treatments were allowed to incubate for 24 or 48 hours. Four genotypes representing two heterotic groups were tested with replication. The effect on potting rate from in vitro culture was evaluated.

Initial testing measured the effect of TMX at concentrations of 0, 150, or 750 mg/L for 24 or 48 hours. The effect of co-treatment with TMX on potting rate showed an overall higher potting rate (80%+) as compared to the control at the 24-hour duration. The strongest effect of TMX co-treatment seen with the 48-hour duration was at 750 mg/L TMX compared to the control. Seedling height from the same experiment was taken as a measure of plant health and showed overall plant height to be taller after the co-treatment of TMX at 150 or 750 mg/L at both 24-hour and 48-hour durations.

Further testing was done and TMX was applied to isolated embryos at levels of 0, 750, 1500, 2000 mg/L during colchicine co-treatment. The co-treatments were incubated for 24 or 48 hours. The 24-hour co-treatment at 1500 and 2000 mg/L resulted in the highest overall percent potted rate, as well as highest percent transplanted rate (See Table 6).

TABLE 6
Co-treatment of embryos with colchicine and 0-2000 mg/L TMX for
24-48 hours: effects on potted rate and transplanted rate.

| Duration | TMX | Potted Rate | | Transplant Rate | |
|---|---|---|---|---|---|
| (hours) | (mg/L) | Mean | Std Error | Mean | Std Error |
| 24 | 0 | 51% | 10% | 31% | 7% |
| | 750 | 52% | 6% | 33% | 6% |
| | 1500 | 65% | 6% | 41% | 8% |
| | 2000 | 74% | 5% | 50% | 7% |
| 48 | 0 | 7% | 3% | 5% | 3% |
| | 750 | 12% | 2% | 9% | 2% |
| | 1500 | 18% | 5% | 12% | 4% |
| | 2000 | 32% | 9% | 21% | 8% |

TMX was applied to isolated embryos at levels of 0, 2000, 4000, 8000 mg/L during colchicine co-treatment. The co-treatments were incubated for durations of 24 or 48 hours. The 24-hour co-treatment at a level of 4000 mg/L TMX resulted in the highest overall percent potted rate and transplanted rate (See Table 7).

TABLE 7
Co-treatment of embryos with colchicine and 0-8000 mg/L TMX
for 24-48 hours: effects on potted rate and transplanted rate

| | | | |
|---|---|---|---|
| Duration | TMX | Potted Rate | Transplant Rate |

| (hours) | (mg/L) | Mean | Std Err | Mean | Std Err |
|---|---|---|---|---|---|
| 24 | 0 | 66% | 5% | 50% | 9% |
| | 2000 | 76% | 14% | 73% | 11% |
| | 4000 | 82% | 6% | 79% | 3% |
| | 8000 | 47% | 6% | 45% | 8% |
| 48 | 0 | 18% | 7% | 16% | 7% |
| | 2000 | 22% | 19% | 15% | 13% |
| | 4000 | 67% | 4% | 61% | 7% |
| | 8000 | 20% | 20% | 16% | 16% |

TMX was applied to isolated embryos at levels of 0, 2000, 4000, or 6000 mg/L during colchicine co-treatment. The co-treatments were incubated for durations of 24 or 48 hours. The 24-hour co-treatment at 4000 mg/L and 48-hour co-treatment at 6000 mg/L resulted in the highest percent potted rates of 90% and 89%, respectively. The highest percent transplanted rates were 86% and 83% for co-treatments 4000 mg/L at 24 hours and 6000 mg/L at 48 hours, respectively (see Table 8).

TABLE 8
Co-treatment of embryos with colchicine and 0-6000 mg/L TMX
for 24-48 hours: effects on potted rate and transplanted rate

| | | | |
|---|---|---|---|
| Duration | TMX | Potted Rate | Transplant Rate |

| (hours) | (mg/L) | Mean | Std Err | Mean | Std Err |
|---|---|---|---|---|---|
| 24 | 0 | 48% | 8% | 44% | 7% |
| | 2000 | 68% | 4% | 62% | 5% |
| | 4000 | 90% | 4% | 86% | 5% |
| | 6000 | 86% | 8% | 74% | 9% |
| 48 | 0 | 6% | 2% | 6% | 2% |
| | 2000 | 27% | 10% | 26% | 10% |
| | 4000 | 63% | 8% | 56% | 8% |
| | 6000 | 89% | 4% | 83% | 6% |

Higher escape rates were observed with TMX co-treatment of 6000 or 8000 mg/L at 24 hours. Levels of TMX in the range of 2000-6000 mg/L generated the highest potting rates while having reliable marker visibility for selecting haploids due to low escape rates (see Table 9).

TABLE 9
Co-treatment of embryos with colchicine and 0-8000
mg/L TMX for 24-48 hours: effects on escape rate

| TMX (mg/L) | 48 hr Escape Rate | 24 hr Escape Rate |
|---|---|---|
| Control | 0% | 0% |
| 2000 | 0% | 0% |
| 4000 | 0% | 8% |
| 6000 | 0% | 60% |
| 8000 | 33% | 50% |

### Example 7. Thiamethoxam Liquid Media Pre-Treatment Prior to Colchicine Doubling

TMX pre-treatment application to isolated embryos prior to colchicine doubling was done in 2 genotypes with replication. Isolated embryos were plated on filter paper and pre-treated with a formulation of TMX at concentrations ranging from 10-1000 mg/L in liquid media. The pre-treatments incubated for 0.5 to 24 hours. Following the pre-treatment, embryos were treated with colchicine for chromosome doubling and plant regeneration. The effect on potting rate and transplant rate were evaluated (see Table 10).

None of the liquid pre-treatments resulted in higher potting or transplant rates compared to the solid media control, but the 1 hr, 1000 mg/L pre-treatment came closest reaching a potting rate of 58% and transplant rate of 55% compared to rates of 65% and 64%, respectively, in the solid media control. Liquid media may not provide an improvement on its own, but use of TMX to improve embryo potting and transplant rates in a liquid media would be useful in a production environment where liquid handling provided other advantages in throughput or lower cost of goods.

TABLE 10
Pre-treatment of embryos with 0-1000 mg/L TMX for
1-24 hours: effect on potting and transplant rates

Media &

| Duration | | Potting Rate | | Transplant Rate | |
|---|---|---|---|---|---|
| (hours) | TMX (mg/L) | Mean | Std Dev | Mean | Std Dev |
| Solid | 0 | 65% | 6% | 64% | 8% |
| Media | | | | | |
| (Control) | | | | | |
| 1 hr | 0 | 37% | 3% | 34% | 4% |
| Liquid | 10 | 55% | 5% | 51% | 5% |
| Media | 100 | 38% | 3% | 37% | 3% |
| | 1000 | 58% | 6% | 55% | 7% |
| 24 hr | 0 | 6% | 2% | 6% | 2% |
| Liquid | 10 | 7% | 2% | 7% | 2% |
| Media | 100 | 15% | 5% | 15% | 5% |
| | 1000 | 11% | 2% | 11% | 2% |

In a follow-up experiment, the liquid pre-treatment was tested at 30 minute and 1-hour durations to determine if a shorter duration would result in better potting and transplant rates than observed in the 1 hour treatments shown in Table 10. These were performed in the same manner as described above, in triplicate. The results showed that a 30-minute liquid media pre-treatment was not superior to a 1 hour (see Table 11).

TABLE 11
Pre-treatment of embryos with 0-1000 mg/L TMX for 30
min-1 hour: effect on potting and transplant rates

| Duration | | Potting Rate | | Transplant Rate | |
|---|---|---|---|---|---|
| (hours) | TMX (mg/L) | Mean | Std Error | Mean | Std Error |
| Solid | 0 | 43% | 4% | 38% | 4% |
| Media | | | | | |
| Control | | | | | |
| 30 min | 100 | 68% | 8% | 54% | 6% |
| Liquid | 1000 | 51% | 2% | 42% | 1% |
| 1 hr | 100 | 77% | 11% | 66% | 8% |
| Liquid | 1000 | 74% | 3% | 56% | 7% |

### Example 8. Thiamethoxam Post-Treatment after Colchicine Doubling (During Germination)

TMX was applied to doubled haploids by adding it to the germination media during the germination process. TMX was tested in 3 genotypes, in triplicate, with concentrations ranging from 0-12 g/L, and the post-treatments incubated for 14 to 21 days (the normal duration of the regeneration period before transplant). The effect on potting rate was evaluated (see Table 12); no plants were advanced for transplant rate assessments due to poor germination.

TABLE 12
Germination post-treatment of embryos with 0-12
g/L TMX for 14-21 days: effect on potting rates

Potting Rate

| TMX | | Std |
|---|---|---|
| (g/L) | Mean | Dev |
| 0 | 58% | 2% |
| 0.5 | 53% | 1% |
| 1 | 50% | 5% |
| 1.5 | 27% | 4% |
| 5 | 3% | 1% |
| 12 | 0% | 0% |

### Example 9. Co-Treatment of Isolated Embryos with Metcamifen and Thiamethoxam, Including Growth to Maturity, Self-Pollination, and DH1 Seed Set Evaluation

To assess the impact of the safener and vigor enhancing compounds on the full DH production pipeline, experiments were designed and conducted spanning haploid embryo isolation, doubling treatment, germination, potting, growth to maturity, selfing, and seed set evaluation. Embryos from maize ears of two genotypes were isolated and co-treated with both metcamifen and thiamethoxam during the colchicine doubling treatment, in triplicate. Metcamifen was added in the Murashige and Skoog Medium (MS medium) at concentrations ranging from 400 to 800 mg/L in combination with TMX at concentrations ranging from 4000 to 6000 mg/L. Control co-treatments with metcamifen alone, TMX alone, or colchine-only were run in parallel. Concentrations for co-treatments were selected based on best outcomes from experiments in Examples 1-8. The co-treatments incubated for 24 and 48 hours at 28° C. with 150 PAR light. Resulting embryos were germinated and the percentage of embryos successfully transferred to soil was measured as the potting rate. The survival of healthy plants was measured as the transplant rate.

Transplanted seedlings were grown to maturity and self-pollinated to assess the rate at which fertile DH plants resulted in ears with greater than 4 and greater than 49 kernels per ear (ears >4 and ears >49 rates). The highest potting (96%) and transplant rates (95%) were achieved in the 24-hour co-treatments with 400 mg/L of metcamifen and 4000 mg/L TMX combined but were not statistically significantly different than the rates observed for 4000 mg/L TMX without metcamifen (p<0.0001). All co-treatments were significantly better (p<0.0001) than the colchicine control at both timepoints for potting and transplant rates. Across all co-treatments, and the colchicine-only treatment control, the 48-hour experiment resulted in overall lower potting and transplant rates (12-69% and 10-64%, respectively) than observed in the corresponding 24-hour experiments (58%-96% and 48-95%, respectively) (see Table 13). This is likely due to the higher toxicity of colchicine exposure with longer durations.

The rate of ears setting more than 4 kernels (ears >4 rate) for all co-treatments were significantly increased (p<0.005) compared to the colchicine-only treatment control for both the 24- and 48-hour co-treatment groups, but no significant differences were observed amongst the co-treatments (see Table 13). For the rate of ears setting more than 49 kernels (ears >49 rate), there was no statistically significant difference between any of the co-treatments and the colchicine-only treatment control at the 24-hour timepoint. For the 48-hour co-treatment, the metcamifen combined with TMX resulted in a higher probability of producing an ear with >49 kernels than either metcamifen or TMX co-treatment alone.

TABLE 13
Co-treatment of embryos with colchicine and 0-6000 mg/L TMX,
and/or 0-800 mg/L metcamifen for 24 or 48 hours: effects on
potted rate, transplanted rate, and ear kernel setting rates

| | | | | | Transplant | | | | | |
|---|---|---|---|---|---|---|---|---|---|---|
| | | | Potted Rate | | Rate | | Ears >4 Rate | | Ears >49 Rate | |
| Duration | Metcamifen | TMX | | Std | | Std | | Std | | Std |
| (hours) | (mg/L) | (mg/L) | Mean | Err | Mean | Err | Mean | Err | Mean | Err |
| 24 | 0 | 0 | 58% | 4% | 48% | 6% | 25% | 4% | 16% | 3% |
| | 800 | 0 | 76% | 4% | 68% | 6% | 38% | 4% | 24% | 4% |
| | 0 | 6000 | 95% | 2% | 93% | 2% | 45% | 4% | 24% | 4% |
| | 400 | 4000 | 96% | 2% | 95% | 2% | 48% | 4% | 18% | 3% |
| 48 | 0 | 0 | 12% | 3% | 10% | 2% | 6% | 2% | 4% | 2% |
| | 800 | 0 | 15% | 3% | 15% | 3% | 9% | 2% | 4% | 2% |
| | 0 | 4000 | 79% | 3% | 77% | 3% | 30% | 4% | 13% | 3% |
| | 800 | 4000 | 69% | 4% | 64% | 4% | 36% | 4% | 24% | 4% |

Escapes were recorded at two steps in the experiment. The first record was taken in the lab when embryos failed to change color until after moving from doubling media to germination media. The second record was taken in the greenhouse when plants presented purple coloring or other obvious diploid characteristics (tall height, leaf width, large quantities of fertile pollen, purple kernels at seed maturity). As was observed in the experiment in Example 6 (see Table 9), co-treatment with TMX resulted in a higher number of escapes. This was also true when TMX was combined with metcamifen for embryo co-treatment, although the 24-hour metcamifen co-treatment group also had 24% escapes detected in the greenhouse in the absence of TMX. In the two genotypes tested, one line had much higher escape rates than the other (see Table 14).

TABLE 14
Co-treatment of embryos with colchicine and 0-6000 mg/L TMX, and
0-800 mg/L metcamifen for 24-48 hours: effects on escape rate

| | | | Genotype 1 | | | Genotype 2 | | |
|---|---|---|---|---|---|---|---|---|
| | Metcamifen | TMX | Lab | GH | | Lab | GH | |
| | (mg/L) | (mg/L) | Escapes | Escapes | Total | Escapes | Escapes | Total |
| 48 | 0 | 0 | 0% | 5% | 5% | 0% | 0% | 0% |
| Hour | 800 | 0 | 0% | 5% | 5% | 0% | 0% | 0% |
| | 0 | 6000 | 9% | 9% | 17% | 0% | 0% | 0% |
| | 400 | 4000 | 0% | 18% | 18% | 0% | 0% | 0% |
| 24 | 0 | 0 | 0% | 13% | 13% | 0% | 0% | 0% |
| Hour | 800 | 0 | 0% | 24% | 24% | 0% | 0% | 0% |
| | 0 | 4000 | 3% | 16% | 19% | 1% | 0% | 1% |
| | 800 | 4000 | 3% | 13% | 15% | 10% | 0% | 10% |

One way of measuring the success of DH production is a ratio of output:input, defined as “DH yield”, where a larger ratio is typically desirable for a more efficient production system. In this study, we measured this in two ways: DH ears/total haploid embryos and DH ears/transplants (see Table 15). For these data, in the 24-hour co-treatment groups, DH yields of ears with >4 kernels increased significantly on a per haploid basis with metcamifen, TMX, and metcamifen+TMX co-treatments compared to the colchicine only control. No significant changes to DH yield were observed in the 24-hour co-treatments for ears >4 kernels on a per transplant basis or for ears >49 kernels on a per haploid basis, although there was a trend towards increased DH yield for the >49 kernels on a per haploid basis comparison for the metcamifen co-treatment. Interestingly, there was a significant negative trend in DH yield for ears >49 kernels on a per transplant basis in co-treatments that included TMX, perhaps indicating that TMX co-treatments increase the potting and transplant rates but that not all those transplants produce ears with higher numbers of kernels.

For these data, in the 48-hour co-treatment groups, we generally saw positive trends associated with all treatments, but the data was challenging to interpret quantitatively because no control plants successfully made ears with kernels. This is likely due to the stress of the additional time spent on colchicine-containing media during the 48-hour duration co-treatment; excessive colchicine exposure is known to cause sterility for both male and female reproductive systems in plants.

TABLE 15
Co-treatment of embryos with colchicine and 0-6000 mg/L TMX, and
0-800 mg/L metcamifen for 24-48 hours: effects on ratios of DH
yield for total haploids and transplants compared to ear rates

| | | | Ears >4 DH Yield | | | | Ears >49 DH Yield | | | |
|---|---|---|---|---|---|---|---|---|---|---|
| | | | DH | | DH | | DH | | DH | |
| | | | Ears/Total | | Ears/ | | Ears/Total | | Ears/ | |
| | | | Haploids | | Transplants | | Haploids | | Transplants | |
| Duration | Metcamifen | TMX | | Std | | Std | | Std | | Std |
| (hours) | (mg/L) | (mg/L) | Mean | Err | Mean | Err | Mean | Err | Mean | Err |
| 24 | 0 | 0 | 0.23 | 0.03 | 0.47 | 0.06 | 0.15 | 0.03 | 0.31 | 0.05 |
| | 800 | 0 | 0.33 | 0.04 | 0.47 | 0.05 | 0.21 | 0.03 | 0.30 | 0.04 |
| | 0 | 6000 | 0.41 | 0.04 | 0.44 | 0.04 | 0.20 | 0.03 | 0.21 | 0.04 |
| | 400 | 4000 | 0.45 | 0.04 | 0.47 | 0.04 | 0.15 | 0.03 | 0.16 | 0.03 |
| 48 | 0 | 0 | 0.00 | — | 0.00 | — | 0.00 | — | 0.00 | — |
| | 800 | 0 | 0.07 | 0.02 | 0.61 | 0.15 | 0.03 | 0.02 | 0.25 | 0.12 |
| | 0 | 4000 | 0.29 | 0.04 | 0.37 | 0.04 | 0.12 | 0.03 | 0.16 | 0.03 |
| | 800 | 4000 | 0.33 | 0.04 | 0.51 | 0.05 | 0.22 | 0.03 | 0.33 | 0.05 |

### Example 10. Metcamifen and Thiamethoxam Co-Treatment During Colchicine Doubling in Liquid Media Versus Medium Containing Gelzan

Isolated embryos were placed in a Petri dish on filter paper wetted with liquid Murashige and Skoog Medium (MS medium) containing colchicine and a formulation of metcamifen at 800 mg/L, TMX at 4000 mg/L, or a combination of 800 mg/L metcamifen and 4000 mg/L TMX. The colchicine treatment control used the same conditions except with a medium containing 3.5 g/L Gelzan as compared to the liquid medium without Gelzan. The co-treatments incubated for 24 or 48 hours at 28° C. with 150 PAR light and were conducted on 2 genotypes with 2 replicates. The effects on GST expression, potting rate, and transplant rate are reported (see Table 16). The rate of diploid escapes observed increased to 6.4-8.5% in the 24-hour co-treatments with 4000 mg/L TMX and 4000 mg/L TMX combined with 800 mg/L metcamifen. In all other co-treatments, the escape rate was 0%. All 24-hour duration co-treatments resulted in high potting rates (91.2-92.5%) compared to the control (78%), and higher than the 48-hour co-treatments (22-82%). The best transplant rates observed were in the 24-hour duration 4000 mg/L TMX co-treatment (90%) compared to the control (72%).

TABLE 16
Co-treatment of embryos with metcamifen 0-800 mg/L and TMX
0-4000 mg/L for 24-48 hours in liquid culture medium: effect
on GST expression, potting, and transplant rates

| | | | GST27 | | | | Transplant | | |
|---|---|---|---|---|---|---|---|---|---|
| | | | Induction | | Potting Rate | | Rate | | |
| | Metcamifen | TMX | (ng/mg TSP) | | | St | | St | Escape |
| | (mg/L) | (mg/L) | Before | After | Mean | Dev | Mean | Dev | Rate |
| 48 | 0 | 0 | 0 | 200 | 3.6% | 1.5 | 2.4% | 1.0 | 0% |
| Hour | 800 | 0 | 0 | 396 | 21.5% | 6.9 | 20.4% | 6.4 | 0% |
| | 0 | 4000 | 1 | 194 | 79.4% | 6.9 | 72.2% | 5.7 | 0% |
| | 800 | 4000 | 2 | 480 | 82.4% | 4.4 | 74.1% | 5.2 | 0% |
| 24 | 0 | 0 | 0 | 177 | 77.5% | 7.8 | 71.9% | 8.2 | 0% |
| Hour | 800 | 0 | 0 | 270 | 92.5% | 5.3 | 88.2% | 5.4 | 0% |
| | 0 | 4000 | 0 | 255 | 92.3% | 9.3 | 89.7% | 9.0 | 8.5% |
| | 800 | 4000 | 3 | 444 | 91.2% | 1.7 | 67.0% | 1.2 | 6.4% |

In a separate experiment, additional concentrations of metcamifen and TMX co-treatment were tested in liquid co-treatments. The experimental setup was the same as described above for the data in Table 16, but the co-treatment concentrations were varied. The colchicine-only control remained unchanged, the metcamifen co-treatment was maintained at 800 mg/L, the TMX co-treatment was increased to 6000 mg/L in the 48-hour co-treatment and maintained at 4000 mg/L in the 24-hour co-treatment, and the combination of metcamifen with TMX co-treatment decreased the metcamifen concentration to 400 mg/L with 4000 mg/L TMX. The decreased concentrations were associated with lower rates of escapes in the 24-hour co-treatments, only the metcamifen with TMX combination co-treatment produced escapes (1.8%). Potting and transplant rates remained high, with the best performance in the 24-hour metcamifen with TMX combination co-treatment (96% potting, and 87% transplant) compared to the colchicine-only control (60% potting, and 47% transplant) (see Table 17). In all liquid media co-treatments, higher rates of bacterial and/or fungal contamination were observed. In total, 35% of the attempted experiments were discarded due to contamination issues and excluded from the data analysis.

TABLE 17
Co-treatment of embryos with metcamifen 0-800 mg/L and TMX
0-6000 mg/L for 24-48 hours in liquid culture medium: effect
on GST expression, potting, and transplant rates

| | | | GST27 | | | | Transplant | | |
|---|---|---|---|---|---|---|---|---|---|
| | | | Induction | | Potting Rate | | Rate | | Escape |
| | Metcamifen | TMX | (ng/mg TSP) | | | St | | St | Rate |
| | (mg/L) | (mg/L) | Before | After | Mean | Dev | Mean | Dev | (%) |
| 48 | 0 | 0 | 5 | 139 | 37.7% | 10.7 | 37.7% | 10.7 | 0.0 |
| Hour | 800 | 0 | 4 | 340 | 9.2% | 2.1 | 9.2% | 2.1 | 0.0 |
| | 0 | 6000 | 13 | 208 | 58.0% | 3.1 | 54.0% | 2.6 | 0.0 |
| | 400 | 4000 | 8 | 408 | 80.7% | 3.8 | 73.7% | 3.6 | 0.0 |
| 24 | 0 | 0 | 3 | 163 | 59.7% | 9.1 | 46.8% | 7.8 | 0.0 |
| Hour | 800 | 0 | 0 | 263 | 86.2% | 6.7 | 74.1% | 6.7 | 0.0 |
| | 0 | 4000 | 5 | 193 | 77.3% | 5.8 | 81.3% | 7.2 | 0.0 |
| | 400 | 4000 | 5 | 265 | 96.4% | 4.2 | 87.3% | 3.6 | 1.8 |

### Example 11. Co-Treatment with Safeners Benoxacor or Dichlormid on Isolated Embryos During Colchicine Treatment

Benoxacor was applied to isolated embryos of 2 genoytypes, with 2 replicates at levels of 0, 20, 50, 100 or 200 mg/L during colchicine treatment in the doubling media. The co-treatments were incubated for durations of 24 or 48 hours. The 24-hour co-treatment at 50 mg/L resulted in the highest percent potting rate of 90%. None of the 48-hour co-treatments performed better than the equivalent co-treatment at 24 hours (see Table 18). Escape rate was not directly measured in these experiments, but there was a mild decrease in color expression (that is the portion of the diploid embryos visibly displaying purple pigment) correlated with increasing concentration of benoxacor in the 48-hour co-treatment.

TABLE 18
Co-treatment of embryos with colchicine and 0-200 mg/L
of benoxacor for 24-48 hours: effects on potting rate

Potting Rate

| Duration (hours) | Benoxacor (mg/L) | Mean | Std Err |
|---|---|---|---|
| 24 | 0 | 76% | 8% |
| | 20 | 79% | 18% |
| | 50 | 90% | 8% |
| | 100 | 79% | 12% |
| 48 | 0 | 10% | 5% |
| | 50 | 40% | 22% |
| | 100 | 60% | 21% |
| | 200 | 74% | 14% |

Dichlormid was applied to isolated embryos from 2 genotypes, with 2 replicates at levels of 0, 20, 50, 100, 200 and 300 mg/L during colchicine treatment in the doubling media. The co-treatments were incubated for durations of 24 or 48 hours. The 24-hour co-treatments at 100 mg/L and 50 mg/L resulted in the highest percent potted rates of 96% and 90%, respectively. The highest percent transplanted rates were 92% and 88% for co-treatments 100 mg/L and 50 mg/L both at 24 hours, respectively. None of the 48-hour co-treatments performed better than the equivalent co-treatment at 24 hours (see Table 19). Escape rate was not directly measured in these experiments, but there was a steep decrease in color expression (that is the portion of the diploid embryos colored purple) correlated with concentrations greater than 100 mg/L.; this likely increases risk of escapes at higher rates of co-treatment.

TABLE 19
Co-treatment of embryos with colchicine and 0-300 mg/L of dichlormid
for 24-48 hours: effects on potted and transplant rate

| Duration | Dichlormid | Potting Rate | | Transplant Rate | |
|---|---|---|---|---|---|
| (hours) | (mg/L) | Mean | Std Err | Mean | Std Err |
| 24 | 0 | 85% | 8% | 81% | 8% |
| | 20 | 89% | 3% | 86% | 4% |
| | 50 | 91% | 1% | 89% | 2% |
| | 100 | 97% | 1% | 92% | 3% |
| 48 | 0 | 6% | 4% | 6% | 4% |
| | 100 | 70% | 17% | 70% | 17% |
| | 200 | 86% | 6% | 84% | 7% |
| | 300 | 95% | 2% | 93% | 2% |

### Example 12. Hohenheim Corn DH Method (Deimling et al., 1997; Prigge and Melchinger, 2012) for Applying Metcamifen and TMX on R1-nj Mature Seeds

Germinated haploid seedings may be co-treated with metcamifen and TMX during the colchicine doubling treatment. Metcamifen is added in the colchicine treatment solution at concentrations ranging from 80 to 800 mg/L in combination with TMX at concentrations ranging from 400 to 4000 mg/L. The co-treatments incubate for 6-12 hours. The co-treated seedlings are rinsed with water before being transplanted to soil and grown to maturity for doubled haploid seed production.

### REFERENCES

Ian Jepson, et al. 1994. Cloning and characterization of maize herbicide safener-induced cDNAs encoding subunits of glutathione S-transferase isoforms I, II and IV. Plant Molecular Biology 26:1855-1866.
David Holt, Ian Jepson, et al. 1995. Characterization of the safener-induced glutathione S-transferase isoform II from maize. Planta 196:295-302
Riechers D E, Kreuz K, Zhang Q. 2010. Detoxification without intoxication: herbicide safeners activate plant defense gene expression. Plant Physiology 153, 3-13.
Melissa Brazier-Hicks, Gavin Hall et al. 2020. Chemically induced herbicide tolerance in rice by the safener Metcamifen is associated with a phased stress response. Journal of Experimental Botany, Vol. 71, No. 1 pp. 411-421.
Deimling S, Röber F, Geiger H H. Methodik und Genetik der in vivo-Haploideninduktion bei Mais. Vorträge für Pflanzenzüchtung. 1997; 38:203-224.
Prigge V, Melchinger A E (2012) Production of haploids and doubled haploids in maize. In: Loyola-Vargas V M, Ochoa-Alejo N (eds) Plant cell culture protocols. Springer, Berlin, pp 161-172

What is claimed is:

1. A method of generating a doubled haploid plant comprising
a) Obtaining a haploid plant tissue,
b) Contacting the haploid plant tissue with a safener compound,
c) Contacting the haploid plant tissue with a chromosome doubling agent, and
d) Regenerating a doubled haploid plant, wherein the safener compound and the chromosome doubling agent applied sequentially or simultaneously.

2. (canceled)

3. The method of claim 1, wherein the haploid plant tissue is further contacted with a vigor response compound, wherein the vigor response compound and chromosome doubling agent are applied sequentially or simultaneously.

4. (canceled)

5. The method of claim 1, wherein the safener compound is contacted with the haploid plant tissue prior to step c) for 24 hours to 168 hours.

6. (canceled)

7. The method of claim 5, wherein the safener compound is contacted with the haploid plant tissue at a concentration of 5-50 mg/L.

8. The method of claim 7, wherein the safener compound contact with the haploid plant tissue occurs at 4-8° C.

9. (canceled)

10. The method of claim 1, wherein the safener compound is contacted with the haploid plant tissue concurrently with step c) for 24 hours to 48 hours.

11. (canceled)

12. The method of claim 10, wherein the safener compound is contacted with the haploid plant tissue at a concentration of 400-800 mg/L.

13. The method of claim 12, wherein the safener compound contact with the haploid plant tissue occurs at 24-32° C.

14. (canceled)

15. The method of claim 1, wherein the safener compound is contacted with the haploid plant tissue after step c) for 8-504 hours.

16. (canceled)

17. The method of claim 15, wherein the safener compound contact with the haploid plant tissue is at a concentration of 0-20 mg/L.

18. The method of claim 17, wherein the safener compound contact with the haploid plant tissue occurs at 24-32° C.

19. (canceled)

20. The method of claim 1, wherein the safener compound and the vigor response compound are applied sequentially or simultaneously.

21. The method of claim 1, wherein the haploid plant tissue is from maize, wheat, rice, barley, sunflower, soybean, watermelon, cucumber, tomato, pepper or brassica.

22. (canceled)

23. The method of claim 21, wherein the maize haploid plant tissue is an ear, a microspore, callus tissue, or an embryo.

24. (canceled)

25. The method of claim 1, wherein the safener compound is selected from the group consisting of metcamifen, benoxacor, dichlormid, isoxadifen, cloquintocet-mexyl, fenclorim, and cyprosulfamide.

26. (canceled)

27. The method of claim 1, wherein the chromosome doubling agent is selected from the group consisting of colchicine, trifluralin, pronamide, dithipyr, nitrous oxide, and oryzalin.

28. (canceled)

29. The method of claim 3, wherein the vigor response compound is selected from the group consisting of thiamethoxam and saponins.

30. (canceled)

31. The method of claim 29, wherein the haploid plant tissue is contacted with thiamethoxam prior to colchicine treatment, during the colchicine treatment, or after the colchicine treatment.

32. The method of claim 31, wherein the thiamethoxam is applied to the haploid plant tissue prior to the colchicine treatment at a concentration of 10-1000 mg/L.

33. The method of claim 32, wherein the thiamethoxam is applied to the haploid tissue prior to the colchicine treatment for 24 to 48 hours.

34. (canceled)

35. The method of claim 31, wherein the thiamethoxam is applied to the haploid plant tissue during the colchicine treatment at a concentration of 100 to 8000 mg/L.

36. The method of claim 35, wherein the thiamethoxam is applied to the haploid plant tissue during the colchicine treatment for 24 to 48 hours.

37. (canceled)

38. The method of claim 31, wherein the thiamethoxam is applied to the haploid plant tissue after the colchicine treatment at a concentration of 0.5-12 g/L.

39. The method of claim 38, wherein the thiamethoxam is applied to the haploid plant tissue after the colchicine treatment for 24-504 hours.

40. The method of claim 1, wherein the contact is performed in solid or liquid media.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)
